(19) United States
(12) Patent Application Publication
(10) Pub. No.: US 2026/0027033 A1
(43) Pub. Date: Jan. 29, 2026

(54) BIODEGRADABLE CELLULOSE ESTER MICROPARTICLES AND SYSTEMS AND METHODS FOR THE PRODUCTION THEREOF

(71) Applicant: Eastman Chemical Company, Kingsport, TN (US)

(72) Inventors: GLENN LEWIS SHOAF, JONESBOROUGH, TN (US); ROBERT DAVID PACE, CHURCH HILL, TN (US); TIMOTHY R PITTS, KINGSPORT, TN (US); GARRY KENNETH WEAKLEY, KINGSPORT, TN (US); SHARMISTHA MAZUMDER, JONESBOROUGH, TN (US); JUN HU, JOHNSON CITY, TN (US); DEEPIKA SAINI, KINGSPORT, TN (US)

(73) Assignee: Eastman Chemical Company, Kingsport, TN (US)

(21) Appl. No.: 19/099,076

(22) PCT Filed: Mar. 22, 2024
(86) PCT No.: PCT/US2024/021027

§ 371 Date: Jan. 28, 2025

Related U.S. Application Data
(60) Provisional application No. 63/491,626, filed on Mar. 22, 2023.

(51) Int. Cl. A61K 8/73

(52) U.S. Cl. CPC A61K 8/731

(57) ABSTRACT

Systems and methods of producing cellulose ester (CE) microparticles are provided. The CE microparticles are produced by milling initial CE particles, having an average particle size of at least 75 microns, to a D50 particle size in the range of 0.5 to 50 microns. The produced microparticles are biodegradable, and have acceptable solidity and/or tactile feel to be used as an economical substitute for conventional plastic microbeads.

### BACKGROUND

Microbeads are typically spheroidal plastic particles having a diameter of less than 1 millimeter (mm). These particles are sometimes included in consumer products, such as personal care and cosmetic products. Many of these microbead-containing products are designed to be applied and then washed or rinsed from the user's body. When the microbead-containing products are washed or rinsed from the user's body, the particles are flushed down the drain and received at municipal water treatment facilities. In the past, many known microbeads had been formed from plastic or polymeric materials, such as polyethylene, polypropylene, polymethyl methacrylate, nylon, polyurethane, and the like. These materials generally have limited biodegradability. In addition, the small size of the particles limits their ability to be captured at the water treatment facilities, such that the particles may be discharged from the facilities and into larger bodies of water (e.g., rivers, seas, and oceans). Once in these larger bodies of water, the plastic or polymeric microbeads may be ingested by wildlife or cause other environmental concerns. Thus, the possibility of producing microbead particles from more environmentally friendly materials has recently been explored. However, consumers tend to have high expectations when it comes to the personal care and/or cosmetic products they use, including those containing micron-sized particles. For example, consumers expect these personal care and cosmetic products to exhibit certain optical properties that allow the products to blend in with natural skin color and readily hide any undesirable blemishes.

Thus, it is desirable to economically produce biodegradable microparticles that meet environmental and consumer expectation standards, particularly regarding optical properties for personal care and cosmetic products.

### SUMMARY

Provided herein is a method of producing biodegradable cellulose ester (CE) microparticles. The method includes providing initial CE particles having an average particle size of at least 75 microns, and milling the initial CE particles to thereby form CE microparticles having a D50 particle size in the range of 0.5 to 50 microns.

Also provided herein is a system for producing cellulose ester (CE) microparticles. The system includes a source of initial CE particles, a compressor for producing compressed gas, a jet mill for receiving and size-reducing the initial CE particles into CE microparticles, and a feed system for supplying the initial CE particles to the jet mill. The jet mill is supplied with a first portion of the compressed gas from the compressor, and the feed system is supplied with a second portion of the compressed gas from the compressor.

Also provided herein is biodegradable cellulose ester (CE) microparticles comprising at least one CE, wherein the CE microparticles have a D50 particle size in the range of 0.5 to 50 microns, wherein the CE microparticles exhibit at least 50 percent biodegradability at 60 days according to the OECD 301B test method.

### BRIEF DESCRIPTION OF THE FIGURES

FIG. 1 is a schematic diagram of an example process for producing cellulose ester microparticles.

FIG. 2 is a schematic diagram of an example system for producing cellulose ester microparticles.

FIG. 3 is a schematic diagram of an alternative system for producing cellulose ester microparticles with separate coarse and fine product packaging stations.

FIG. 4 is a schematic diagram of an example separator that may be used in the systems shown in FIGS. 3 and 4.

### DETAILED DESCRIPTION

The present disclosure is directed to systems and methods for producing cellulose ester (CE) microparticles. The CE microparticles are produced by milling (e.g., jet milling) a biodegradable CE source material. The milling is performed at process conditions that produce micron-sized CE particles having desirable qualities that make them suitable substitutes for conventional microbeads in certain consumer products. The CE microparticles may exhibit characteristics such as enhanced solidity and/or tactile feel, for example, thereby making the jet-milled microparticles generally indistinguishable from conventional microbeads from a consumer standpoint. These qualities, along with the biodegradability of CE, make the CE microparticles produced herein desirable for use in personal care products, cosmetics, and the like.

The present invention may be understood more readily by reference to the following detailed description and the examples provided therein. It is to be understood that this disclosure is not limited to the specific methods, formulations, and conditions described, as such may vary. It is also to be understood that the terminology used herein is for the purpose of describing particular aspects of the disclosed embodiments only and is not intended to be limiting.

Values may be expressed as “about” or “approximately” a given number. Similarly, ranges may be expressed herein as from “about” one particular value and/or to “about” or another particular value. When such a range is expressed, another aspect includes from the one particular value and/or to the other particular value. Similarly, when values are expressed as approximations, by use of the antecedent “about,” it will be understood that the particular value forms another aspect.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “includes,” and “include” have the same open-ended meaning as “comprising,” “comprises,” and “comprise” provided above.

As used herein, a “mixed cellulose ester” shall denote a cellulose ester having at least two different ester substituents on a single cellulose ester polymer chain.

“Degree of Substitution” is used to describe the average substitution level of the substituents per anhydroglucose unit (“AGU”). Generally, conventional cellulose contains three hydroxyl groups in each AGU that can be substituted. Therefore, the DS can have a value between 0 and 3. However, low molecular weight cellulose mixed esters can have a total degree of substitution slightly above 3 from end group contributions. Low molecular weight cellulose mixed esters are discussed in more detail subsequently in this disclosure. Because DS is a statistical mean value, a value of 1 does not assure that every AGU has a single substituent. In some cases, there can be unsubstituted anhydroglucose units, some with two and some with three substituents, and more often than not the value will be a noninteger. Total DS is defined as the average number of all of substituents per anhydroglucose unit. The degree of substitution per AGU can also refer to a particular substituent, such as, for example, hydroxyl, acetyl, butyryl, or propionyl. Additionally, the degree of substitution can specify a given hydroxyl based on the carbon unit of the anhydroglucose unit.

When the degree of substitution refers to hydroxyl, i.e, DS_{OH}, the reference is to the average hydroxyl groups per anhydroglucose that are not substituted. As a result, DS_{OH} is not used in the calculation of the total degree of substitution.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. For example, if the specification describes a specific temperature of 62° F., such a description provides literal support for a broad numerical range of 25° F. to 99° F. (62° F.+/−37° F.), an intermediate numerical range of 43° F. to 81° F. (62 □F+/−19° F.), and a narrow numerical range of 53° F. to 71° F. (62° F.+/−9° F.). These broad, intermediate, and narrow numerical ranges should be applied not only to the specific values, but should also be applied to differences between these specific values. Thus, if the specification describes a first pressure of 110 psia and a second pressure of 48 psia (a difference of 62 psi), the broad, intermediate, and narrow ranges for the pressure difference between these two streams would be 25 to 99 psi, 43 to 81 psi, and 53 to 71 psi, respectively.

Throughout this application, where patents or publications are referenced, the disclosures of these references in their entireties are intended to be incorporated by reference into this application, to the extent they are not inconsistent with the present invention, in order to more fully describe the state of the art to which the invention pertains.

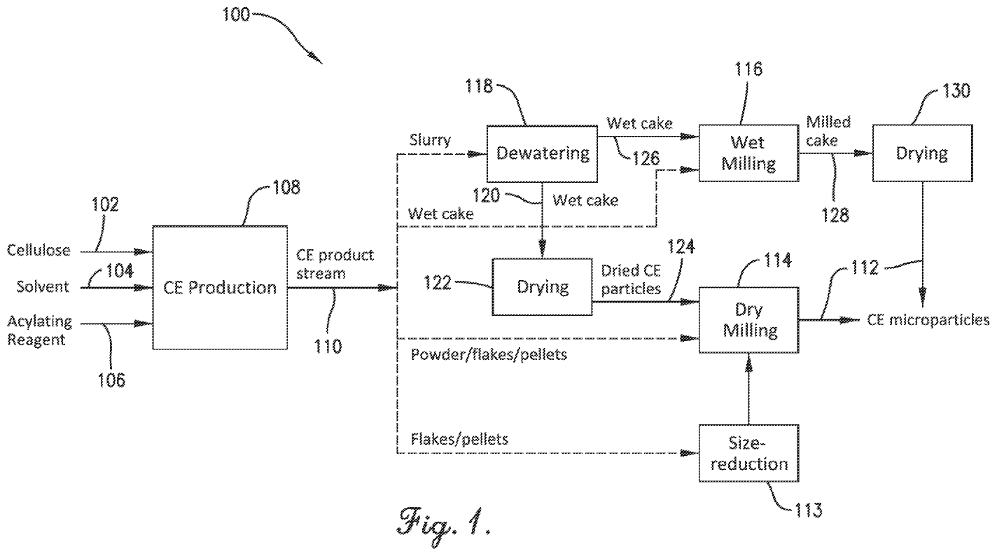

Referring now to FIG. 1, a system 100 is illustrated in which cellulose 102, solvent 104, and an acylating agent 106 are combined at a cellulose ester (CE) production facility 108 to produce a CE product stream 110. CE product stream 110 may be in the form of one or more of a slurry, a wet cake, a powder, flakes, and pellets. The CE in CE product stream 110 is a solid that is subsequently milled to produce CE microparticles 112, as will be described in more detail below.

When the CE product stream 110 is in the form of relatively dry relatively dry (e.g., less than 10 wt. % moisture) powder, flakes, and/or pellets, CE product stream 110 may be channeled directly to a dry milling unit 114 from unit CE production facility 108. The powder, flakes, and/or pellets may then be milled at dry milling unit 114 to a desired average particle size to produce CE microparticles 112. Dry milling unit 114 can be, for example, a jet mill or a mechanical mill. Suitable examples of mechanical mills include ball mills, rod mills, hammer mills, pin mills, and cryogenic mills.

When the CE product stream 110 is in the form of relatively dry (e.g., less than 10 wt. % moisture) flakes and/or pellets, CE product stream 110 may first be size-reduced in a size-reducing unit 113 prior to milling at dry milling unit 114. The flakes and/or pellets may then be milled at dry milling unit 114 to a desired average particle size to produce CE microparticles 112. Examples of suitable size-reducing units 13 include mechanical mills, such as ball mills, rod mills, hammer mills, pin mills, and cryogenic mills.

When the CE product stream 110 is in the form of a slurry (e.g., greater than 50 weight percent liquid), CE product stream 110 may first be dewatered at a dewatering unit 118 to produce a wet cake 120, and wet cake 120 can then be dried at a drying unit 122 to produce dried CE particles 124. Dried CE particles 124 may then be milled at dry milling unit 114 to a desired average particle size to produce CE microparticles 112.

In another embodiment, the slurry from CE production facility 108 is dewatered at dewatering unit 118 to produce a wet cake 126. Wet cake 126 may then be milled at wet milling unit 116 to produce a milled cake 128. Milled cake 128 may then be dried at a drying unit 130 to produce CE microparticles 112 from the solids content of milled cake 128.

When CE product stream 110 is in the form of a wet cake (e.g., 10 to 50 weight percent liquid), CE product stream 110 may be channeled directly to wet milling unit 114 from CE production facility 108. The wet cake may then be milled at wet milling unit 116 to produce milled cake 128. Milled cake 128 may then be dried at unit 130 to produce CE microparticles 112 from the solids content of milled cake 128. Alternatively, the wet cake CE product stream from CE production facility 108 can be routed to drying unit 122 to produce dried CE particles 124. The dried CE particles 124 can then be subjected to dry milling in dry milling unit 114, thereby producing CE microparticles 112.

In one embodiment or in combination with any embodiment mentioned herein, one or more additives can be introduced prior to, during, or after milling in dry milling unit 114 or wet milling unit 116. The additives can include additives that improve milling or additives that enhance the performance of the final CE microparticles. For example, zinc stearate at any point in the process to improve dispersion (i.e., prevent agglomeration) of the CE microparticles in the final formulations. Further, plasticizers may be added at any point in the process to lower the glass transition temperature of the cellulose ester. Finally, after milling, the CE microparticles may be contacted with a surfactant to improve optical characteristics of the CE microparticles. In addition, the milled CE microparticles can be subjected to a spheroidization step after milling to enhance the sphericity of the CE microparticles. Spheroidization can be accomplished by, for example, dropping the CE microparticles through heated air.

### Cellulose Esters

The cellulose ester in CE product stream 110 can be cellulose diacetate (“CDA”), a mixed cellulose ester (“MCE”), or combinations of CDA and one or more MCEs. MCEs include, for example, cellulose acetate butyrate (“CAB”) and cellulose acetate propionate (“CAP”).

Generally, the cellulose esters described herein can be produced, such as at CE production facility 108, by any method known in the art. Examples of processes for producing cellulose esters are taught in Kirk-Othmer, Encyclopedia of Chemical Technology, 5th Edition, Vol. 5, Wiley-Interscience, New York (2004), pp. 394-444, the disclosure of which is incorporated by reference in its entirety. Cellulose (i.e., cellulose 102), the starting material for producing cellulose esters, can be obtained in different grades and from sources such as, for example, cotton linters, softwood pulp, hardwood pulp, corn fiber and other agricultural sources, and bacterial celluloses.

One method of producing cellulose esters is by esterification. In such a method, the cellulose is mixed with the appropriate organic acids, acid anhydrides, and/or catalysts and then converted to a cellulose triester. Ester hydrolysis is then performed by adding a water-acid mixture to the cellulose triester, which can be filtered to remove any gel particles or fibers. Water is added to the mixture to precipitate out the cellulose ester. The cellulose ester can then be washed with water to remove reaction by-products followed by dewatering and drying, as described above.

Acylating reagents suitable for use herein can include, but are not limited to, alkyl or aryl carboxylic anhydrides, carboxylic acid halides, and/or carboxylic acid esters containing the above-described alkyl or aryl groups suitable for use in the acyl substituents of the substituted cellulose esters described herein. Examples of suitable carboxylic anhydrides include, but are not limited to, acetic anhydride, propionic anhydride, butyric anhydride, pivaloyl anhydride, benzoic anhydride, and naphthoyl anhydride. Examples of carboxylic acid halides include, but are not limited to, acetyl, propionyl, butyryl, pivaloyl, benzoyl, and naphthoyl chlorides or bromides. Examples of carboxylic acid esters include, but are not limited to, acetyl, propionyl, butyryl, pivaloyl, benzoyl and naphthoyl methyl esters. In one or more embodiments, the acylating reagent can be one or more carboxylic anhydrides selected from the group consisting of acetic anhydride, propionic anhydride, butyric anhydride, pivaloyl anhydride, benzoyl anhydride, and naphthoyl anhydride.

In various embodiments, the cellulose triesters that are hydrolyzed can have three substituents selected independently from alkanoyls having from 2 to 12 carbon atoms. Examples of cellulose triesters include cellulose triacetate, cellulose tripropionate, cellulose tributyrate, or mixed triesters of cellulose, such as cellulose acetate propionate and cellulose acetate butyrate.

These cellulose triesters can be prepared by a number of methods known to those skilled in the art. For example, cellulose triesters can be prepared by heterogeneous acylation of cellulose in a mixture of carboxylic acid and anhydride in the presence of a catalyst, such as H₂SO₄. Cellulose triesters can also be prepared by the homogeneous acylation of cellulose dissolved in an appropriate solvent such as LiCl/DMAc or LiCl/NMP.

After esterification of the cellulose to the triester, part of the acyl substituents can be removed by hydrolysis or by alcoholysis to give a secondary cellulose ester. Secondary cellulose esters can also be prepared directly with no hydrolysis by using a limiting amount of acylating reagent. This process is particularly useful when the reaction is conducted in a solvent that will dissolve cellulose.

The cellulose esters thus prepared generally comprise the following structure:

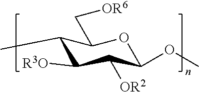

where R², R³, and R⁶ are hydrogen (with the proviso that R², R³, and R⁶ are not hydrogen simultaneously), alkyl-acyl groups, and/or aryl-acyl groups (such as those described above) bound to the cellulose via an ester linkage.

The degree of polymerization (“DP”) of the cellulose esters prepared by these methods can be at least 10. In other embodiments, the DP of the cellulose esters can be at least 50, at least 100, or at least 250. In other embodiments, the DP of the cellulose esters can be in the range of from about 5 to about 100, or in the range of from about 10 to about 50.

The present application discloses, in a first aspect, a mixed ester cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 2.3, the MCE has an average degree of substitution for the propionyl substituents (“DS_{Pr}”) is from 0.1 to 1.2, the MCE has an average degree of substitution for the hydroxyl substituents (“DS_{OH}”) is from 0.6 to 2.8.

In one embodiment or in combination with any other embodiment, class or subclass of this first aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0.Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this first aspect, wherein the DS_{Ac} is from 0.6 to 2.2, or 0.6 to 2.1, or 0.6 to 2.0, or 0.6 to 1.9, or 0.6 to 1.8, or 0.7 to 2.3, or 0.7 to 2.2, or 0.7 to 2.1, or 0.7 to 2.0, or 0.7 to 1.9, or 0.8 to 2.3, or 0.8 to 2.2, or 0.8 to 2.1, or 0.8 to 2.0, or 0.8 to 1.9, or 0.9 to 2.3, or 0.9 to 2.2, or 0.9 to 2.1, or 0.9 to 2.0, or 0.9 to 1.9, or 1.0 to 2.3 or 1.0 to 2.2, or 1.0 to 2.1, or 1.0 to 2.0, or 1.0 to 1.9, or 1.1 to 2.3, or 1.1 to 2.2, or 1.1 to 2.1, or 1.1 to 2.0, or 1.1 to 1.9,, or 1.2 to 2.3 or 1.2 to 2.2, or 1.2 to 2.1, or 1.2 to 2.0, or 1.2 to 1.9, or 0.6 to 1.5, or 0.6 to 1.3, or 0.6 to 1.1, or 0.6 to 0.9 or 0.7 to 1.5, or 0.7 to 1.3, or 0.7 to 1.1, or 0.7 to 0.9.

In one embodiment or in combination with any other embodiment, class or subclass of this first aspect, wherein the DS_{Pr} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4.Additionally, or in the alternative, the DS_{Pr} is less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this first aspect, wherein the DS_{Pr} is from 0.1 to 0.9, or 0.1 to 0.85, or 0.1 to 0.8, or 0.1 to 0.75, or 0.1 to 0.7, or 0.1 to 0.6 or 0.1 to 0.5, or 0.1 to 0.4, or 0.15 to 0.95, or 0.15 to 0.9, or 0.15 to 0.85, or 0.15 to 0.8, or 0.15 to 0.75, or 0.15 to 0.7, or 0.15 to 0.65, or 0.2 to 0.95, or 0.2 to 0.9, or 0.2 to 0.85, or 0.2 to 0.8, or 0.2 to 0.75, or 0.2 to 0.7, or 0.2 to 0.65, 0.25 to 0.95, or 0.25 to 0.9, or 0.25 to 0.85, or 0.25 to 0.8, or 0.25 to 0.75, or 0.25 to 0.7, or 0.25 to 0.65, or 0.3 to 0.95, or 0.3 to 0.9, or 0.3 to 0.85, or 0.3 to 0.8, or 0.3 to 0.75, or 0.3 to 0.7, or 0.3 to 0.65, or 0.35 to 0.95, or 0.35 to 0.9, or 0.35 to 0.85, or 0.35 to 0.8, or 0.35 to 0.75, or 0.35 to 0.7, or 0.35 to 0.65, or 0.4 to 0.95, or 0.4 to 0.9, or 0.4 to 0.85, or 0.4 to 0.8, or 0.4 to 0.75, or 0.4 to 0.7, or 0.4 to 0.65, or 0.45 to 0.95, or 0.45 to 0.9, or 0.45 to 0.85, or 0.45 to 0.8, or 0.45 to 0.75, or 0.45 to 0.7, or 0.45 to 0.65, or 0.5 to 0.95, or 0.5 to 0.9, or 0.5 to 0.85, or 0.5 to 0.8, or 0.5 to 0.75, or 0.5 to 0.7, or 0.5 to 0.65, or 0.1 to 0.9, or 0.1 to 0.85, or 0.1 to 0.8.

In one embodiment or in combination with any other embodiment, class or subclass of this first aspect, wherein the DS_{OH} is at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, or less than 0.8.

In one embodiment or in combination with any other embodiment, class or subclass of this first aspect, wherein the DS_{OH} is from 0.5 to 1.5, or 0.5 to 1.45, or 0.5 to 1.40, or 0.5 to 1.35, or 0.5 to 1.30, or 0.5 to 1.25, or 0.5 to 1.2, or 0.5 to 1.15, or 0.5 to 1.1, or 0.5 to 1.05, or 0.5 to 1.0, or 0.5 to 0.95 or 0.5 to 0.9, or 0.55 to 1.5, or 0.55 to 1.45, or 0.55 to 1.40, or 0.55 to 1.35, or 0.55 to 1.30, or 0.55 to 1.25, or 0.55 to 1.2, or 0.55 to 1.15, or 0.55 to 1.1, or 0.55 to 1.05, or 0.55 to 1.0, or 0.55 to 0.95 or 0.55 to 0.9, or 0.6 to 1.5, or 0.6 to 1.45, or 0.6 to 1.40, or 0.6 to 1.35, or 0.6 to 1.30, or 0.6 to 1.25, or 0.6 to 1.2, or 0.6 to 1.15, or 0.6 to 1.1, or 0.6 to 1.05, or 0.6 to 1.0, or 0.6 to 0.95 or 0.6 to 0.9, or 0.65 to 1.5, or 0.65 to 1.45, or 0.65 to 1.40, or 0.65 to 1.35, or 0.65 to 1.30, or 0.65 to 1.25, or 0.65 to 1.2, or 0.65 to 1.15, or 0.65 to 1.1, or 0.65 to 1.05, or 0.65 to 1.0, or 0.65 to 0.95 or 0.65 to 0.9, or 0.7 to 1.5, or 0.7 to 1.45, or 0.7 to 1.40, or 0.7 to 1.35, or 0.7 to 1.30, or 0.7 to 1.25, or 0.7 to 1.2, or 0.7 to 1.15, or 0.7 to 1.1, or 0.7 to 1.05, or 0.7 to 1.0, or 0.7 to 0.95 or 0.7 to 0.9.

In one embodiment or in combination with any other embodiment, class or subclass of this first aspect, wherein the sum of DS_{Pr} and DS_{Ac} is from 1.9 to 2.44, or 1.9 to 2.0, or 1.9 to 2.1, or 1.9 to 2.2, or 1.9 to 2.3, or 2.0 to 2.44, or 2.0 to 2.1, or 2.0 to 2.2, or 2.0 to 2.3, or 2.1 to 2.44, or 2.1 to 2.2, or 2.1 to 2.3, or 2.2 to 2.44, or 2.2 to 2.3.

In one embodiment or in combination with any other embodiment, class or subclass of this first aspect, wherein the mixed cellulose ester has a ratio of hydroxyl substituents to acetyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this first aspect, wherein the MCE has a ratio of hydroxyl substituents to propionyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, or in the alternative, the MCE has a ratio of hydroxyl substituents to propionyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this first aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any other embodiment, class or subclass of this first aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any other embodiment, class or subclass of this first aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application discloses, in a second aspect, a mixed cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 1.2, the MCE has an average degree of substitution for the propionyl substituents (“DS_{Pr}”) is from 0.1 to 1.4, and the MCE has an average degree of substitution for the hydroxyl substituents (“DS_{OH}”) is from 0.7 to 2.8.

In one embodiment or in combination with any other embodiment, class or subclass of this second aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0.Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this second aspect, wherein the DS_{Ac} is from 0.6 to 0.7, or 0.6 to 0.8, or 0.6 to 0.9, or 0.6 to 1.0, or 0.6 to 1.1, or 0.7 to 0.9, or 0.7 to 1.0, or 0.7 to 1.1, or 0.7 to 1.2, or 0.8 to 0.9, or 0.8 to 1.0, or 0.8 to 1.1, or 0.8 to 1.2, or 0.9 to 1.0, or 0.9 to 1.1, or 0.9 to 1.2, or 1.0 to 1.1, or 1.0 to 1.2, or 1.1 to 1.2.

In one embodiment or in combination with any other embodiment, class or subclass of this second aspect, wherein the DS_{Pr} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. Additionally, or in the alternative, the DS_{Pr} is less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this second aspect, wherein the DS_{Pr} is from 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this second aspect, wherein the DS_{OH} is at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, or less than 0.8.

In one embodiment or in combination with any other embodiment, class or subclass of this second aspect, wherein the DS_{OH} is from 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the mixed cellulose ester has a ratio of hydroxyl substituents to acetyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this second aspect, wherein the MCE has a ratio of hydroxyl substituents to propionyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, or in the alternative, the MCE has a ratio of hydroxyl substituents to propionyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this second aspect, wherein the sum of DS_{Pr} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.3, or 2.0 to 2.2, or 2.0 to 2.1.

In one embodiment or in combination with any other embodiment, class or subclass of this second aspect, wherein the DS_{OH} is from 0.6 to 0.7, or 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any other embodiment, class or subclass of this second aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any other embodiment, class or subclass of this second aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application, in a third aspect, also discloses a mixed cellulose ester (“MCE”), comprising: (1) a plurality of acetyl substituents; (2) a plurality of butyryl substituents; and (3) a plurality of hydroxyl substituents, wherein: the MCE has an average degree of substitution for the acetyl substituents (“DS_{Ac}”) is from 0.1 to 2.4, the MCE has an average degree of substitution for the butyryl substituents (“DS_{Bu}”) is from 0.1 to 1.4, the MCE has an average degree of substitution for the hydroxyl substituents (“DS_{OH}”) is from 0.6 to 2.8.

In one embodiment or in combination with any other embodiment, class or subclass of this first aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0.Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this third aspect, wherein the DS_{Ac} is from 0.9 to 2.4, 0.9 to 2.3, or 0.9 to 2.2, or 0.9 to 2.1, or 0.9 to 2.0, or 0.9 to 1.9, or 0.9 to 1.8, or 0.9 to 1.7, or 0.9 to 1.6, or 0.9 to 1.4, 0.9 to 1.3, or 0.9 to 1.2, or 0.9 to 1.1, or 0.9 to 1.0, or 0.92 to 2.4, 0.92 to 2.3, or 0.92 to 2.2, or 0.92 to 2.1, or 0.92 to 2.0, or 0.92 to 1.9, or 0.92 to 1.8, or 0.92 to 1.7, or 0.92 to 1.6, or 0.92 to 1.4, 0.92 to 1.3, or 0.92 to 1.2, or 0.92 to 1.1, or 0.92 to 1.0, or 0.94 to 2.4, 0.94 to 2.3, or 0.94 to 2.2, or 0.94 to 2.1, or 0.94 to 2.0, or 0.94 to 1.9, or 0.94 to 1.8, or 0.94 to 1.7, or 0.94 to 1.6, or 0.94 to 1.4, 0.94 to 1.3, or 0.94 to 1.2, or 0.94 to 1.1, or 0.94 to 1.0, or 0.96 to 2.4, 0.96 to 2.3, or 0.96 to 2.2, or 0.96 to 2.1, or 0.96 to 2.0, or 0.96 to 1.9, or 0.96 to 1.8, or 0.96 to 1.7, or 0.96 to 1.6, or 0.96 to 1.4, 0.96 to 1.3, or 0.96 to 1.2, or 0.96 to 1.1, or 0.96 to 1.0, or 0.98 to 2.4, 0.98 to 2.3, or 0.98 to 2.2, or 0.98 to 2.1, or 0.98 to 2.0, or 0.98 to 1.9, or 0.98 to 1.8, or 0.98 to 1.7, or 0.98 to 1.6, 0.98 to 1.4, 0.98 to 1.3, or 0.98 to 1.2, or 0.98 to 1.1, or 0.98 to 1.0, or 1.0 to 2.4, 1.0 to 2.3, or 1.0 to 2.2, or 1.0 to 2.1, or 1.0 to 2.0, or 1.0 to 1.9, or 1.0 to 1.8, or 1.0 to 1.7, or 1.0 to 1.6, or 1.0 to 1.4, or 1.0 to 1.3, 1.0 to 1.2, or 1.0 to 1.1, or 1.1 to 2.4, or 1.1 to 2.3, or 1.1 to 2.2, or 1.1 to 2.1, or 1.1 to 2.0, or 1.1 to 1.9, or 1.1 to 1.8, or 1.1 to 1.7, or 1.1 to 1.6, 1.1 to 1.4, or 1.1 to 1.3, or 1.1 to 1.2, or 1.2 to 2.4, or 1.2 to 2.3, or 1.2 to 2.2, or 1.2 to 2.1, or 1.2 to 2.0, or 1.2 to 1.9, or 1.2 to 1.8, or 1.2 to 1.7, or 1.2 to 1.6, or 1.2 to 1.4, or 1.2 to 1.3, or 1.3 to 2.4, or 1.3 to 2.3, or 1.3 to 2.2, or 1.3 to 2.1, or 1.3 to 2.0, or 1.3 to 1.9, or 1.3 to 1.8, or 1.3 to 1.7, or 1.3 to 1.6, or 1.3 to 1.4, or 1.4 to 2.4, or 1.4 to 2.3, or 1.4 to 2.2, or 1.4 to 2.1, or 1.4 to 2.0, or 1.4 to 1.9, or 1.4 to 1.8, or 1.4 to 1.7, or 1.4 to 1.6, or 1.5 to 2.4, or 1.5 to 2.3, or 1.5 to 2.2, or 1.5 to 2.1, or 1.5 to 2.0, or 1.5 to 1.9, or 1.5 to 1.8, or 1.5 to 1.7, or 1.5 to 1.6, or 1.6 to 2.4, or 1.6 to 2.3, or 1.6 to 2.2, or 1.6 to 2.1, or 1.6 to 2.0, or 1.6 to 1.9, or 1.6 to 1.8, or 1.6 to 1.7, or 1.7 to 2.4, or 1.7 to 2.3, or 1.7 to 2.2, or 1.7 to 2.1, or 1.7 to 2.0, or 1.7 to 1.9, or 1.7 to 1.8, or 1.8 to 2.3, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.4, or 2.0 to 2.3, or 2.0 to 2.2, or 2.0 to 2.1, or 2.1 to 2.4, or 2.1 to 2.3, or 2.1 to 2.2, or 2.2 to 2.3.

In one embodiment or in combination with any other embodiment, class or subclass of this third aspect, wherein the DS_{Bu} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. Additionally, or in the alternative, the DS_{Bu} is less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this third aspect, wherein the DS_{Bu} is from 0.1 to 1.35, or 0.1 to 1.3, or 0.1 to 1.25, or 0.1 to 1.2, or 0.1 to 1.15, or 0.1 to 1.1, or 0.1 to 1.0, or 0.1 to 0.8, or 0.1 to 0.6, or 0.2 to 1.35, or 0.2 to 1.3, or 0.2 to 1.25, or 0.2 to 1.2, or 0.2 to 1.15, or 0.2 to 1.1, or 0.2 to 1.0, or 0.2 to 0.8, or 0.2 to 0.6, or 0.2 to 0.4, or 0.3 to 1.35, or 0.3 to 1.3, or 0.3 to 1.25, or 0.3 to 1.2, or 0.3 to 1.15, or 0.3 to 1.1, or 0.3 to 1.0, or 0.3 to 0.8, or 0.3 to 0.6, or 0.3 to 0.5, or 0.4 to 1.35, or 0.4 to 1.3, or 0.4 to 1.25, or 0.4 to 1.2, or 0.4 to 1.15, or 0.4 to 1.1, or 0.4 to 1.0, or 0.4 to 0.8, or 0.4 to 0.6, or 0.5 to 1.35, or 0.5 to 1.3, or 0.5 to 1.25, or 0.5 to 1.2, or 0.5 to 1.15, or 0.5 to 1.1, or 0.5 to 1.0, or 0.5 to 0.8, or 0.5 to 0.7, or 0.6 to 1.35, or 0.6 to 1.3, or 0.6 to 1.25, or 0.6 to 1.2, or 0.6 to 1.15, or 0.6 to 1.1, or 0.6 to 1.0, or 0.6 to 0.8, or 0.7 to 1.35, or 0.7 to 1.3, or 0.7 to 1.25, or 0.7 to 1.2, or 0.7 to 1.15, or 0.7 to 1.1, or 0.7 to 1.0, or 0.8 to 1.35, or 0.8 to 1.3, or 0.8 to 1.25, or 0.8 to 1.2, or 0.8 to 1.15, or 0.8 to 1.1, or 0.8 to 1.0, or 0.9 to 1.35, or 0.9 to 1.3, or 0.9 to 1.25, or 0.9 to 1.2, or 0.9 to 1.15, or 0.9 to 1.1, or 1.0 to 1.35, or 1.0 to 1.3, or 1.0 to 1.25, or 1.0 to 1.2, or 1.0 to 1.15, or 1.0 to 1.1, or 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this third aspect, wherein the DS_{OH} is at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, or less than 0.8.

In one embodiment or in combination with any other embodiment, class or subclass of this third aspect, wherein the DS_{OH} is from 0.5 to 1.0, or 0.5 to 0.95, or 0.5 to 0.9, or 0.5 to 0.85, or 0.5 to 0.8, or 0.5 to 0.75, or 0.5 to 0.7, or 0.5 to 0.65, or 0.5 to 0.6, or 0.5 to 0.55, or 0.55 to 1.0, or 0.55 to 0.95, or 0.55 to 0.9, or 0.55 to 0.85, or 0.55 to 0.8, or 0.55 to 0.75, or 0.55 to 0.7, or 0.55 or 0.65, or 0.55 to 0.6, or 0.6 to 0.65, or 0.6 to 0.7, or 0.6 to 0.75, or 0.6 to 0.8, or 0.6 to 0.85, or 0.6 to 0.9, or 0.6 to 0.95, or 0.6 to 1.0, or 0.65 to 0.7, or 0.65 to 0.75, or 0.65 to 0.8, or 0.65 to 0.85, or 0.65 to 0.9, or 0.65 to 0.95, or 0.65 to 1.0.

In one embodiment or in combination with any other embodiment, class or subclass of this third aspect, wherein the sum of DS_{Bu} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, 2.0 to 2.4, or 2.0 to 2.3, or 2.0 to 2.2, or 2.0 to 2.1.

In one embodiment or in combination with any other embodiment, class or subclass of this third aspect, wherein the mixed cellulose ester has a ratio of hydroxyl substituents to acetyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this third aspect, wherein the mixed cellulose ester has a ratio of hydroxyl substituents to butyryl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, or in the alternative, the mixed cellulose ester has a ratio of hydroxyl substituents to butyryl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this third aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any other embodiment, class or subclass of this third aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

In one embodiment or in combination with any embodiment mentioned herein, the CE that is milled to produce CE microparticles 112 can be the mixed cellulose ester of the first aspect, the second aspect, and/or the third aspect, including any class or subclass of these aspects.

### The Systems

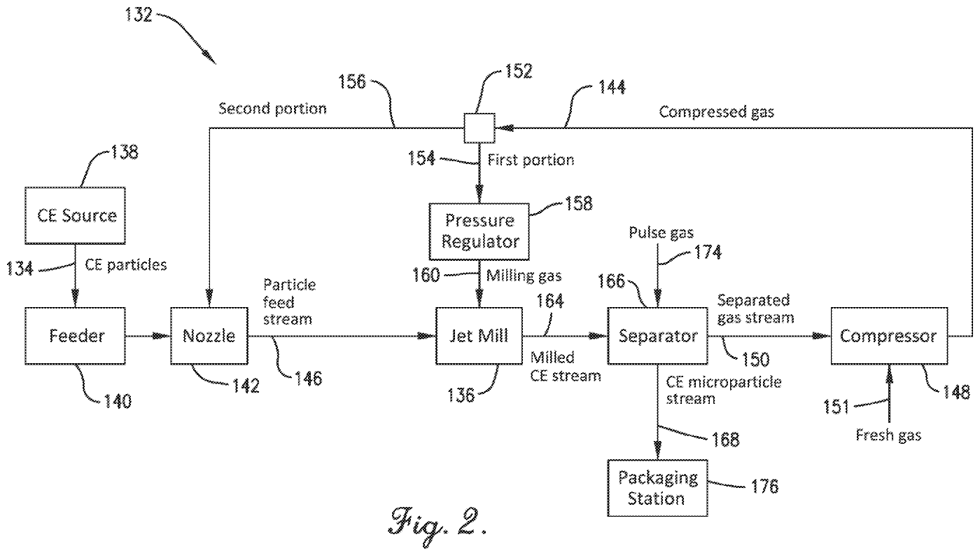

Referring now to FIG. 2, a dry milling system 132 for producing CE microparticles 112 (shown in FIG. 1) is illustrated. In one embodiment, the dry milling system of FIG. 2 is employed as the dry milling unit 114 of FIG. 1. In the embodiment illustrated in FIG. 2, CE particles 134 are provided by a feed system to a jet mill 136 to produce CE microparticles 112 having a desired average particle size. The feed system can include a source 138 of CE particles, a feeder 140 and/or a nozzle 142.

In one embodiment or in combination with any embodiment mentioned herein, source 138 of CE particles includes CE production facility 108 (shown in FIG. 1), as described above. Additionally or alternatively, source 138 of CE particles can be a package, such as a supersack, containing pre-manufactured CE particles 134 received from a CE manufacturer.

CE particles 134 discharged from source 138 are received at feeder 140, which is used to control a feed rate of initial CE particles 134 provided to jet mill 136. Providing CE particles 134 to jet mill 136 at a controlled and substantially steady feed rate enables CE particles 134 to be size-reduced to a desired average particle size. In one embodiment or in combination with any embodiment mentioned herein, feeder 140 is a loss-in-weight feeder.

Before being provided to jet mill 136, CE particles 134 discharged from feeder 140 are received at nozzle 142. CE particles 134 are combined with a portion of a compressed gas 144 (e.g., compressed air), as will be described in more detail below, at nozzle 142 to form a particle feed stream 146. Combining CE particles 134 with compressed gas 144 facilitates conveying CE particles 134 to jet mill 136 in an efficient and controllable manner. In one embodiment or in combination with any embodiment mentioned herein, nozzle 142 is a venturi nozzle. Thus, a pressure differential formed within nozzle 142 by the compressed gas 144 is used to draw CE particles 134 from feeder 140 to nozzle 142 to thereby form particle feed stream 146.

System 132 also includes a compressor 148 for producing compressed gas 144. Compressed gas 144 may be produced by compressor 148 from ambient air and/or from a separated gas stream 150, as will be described in more detail below. A fresh gas stream 151 (e.g., air) may be supplied to compressor 148 when the amount of the separated/recirculated gas stream 150 is insufficient. In one embodiment or in combination with any embodiment mentioned herein, compressed gas 144 discharged from compressor 148 is provided for use in at least one of jet mill 136, or for use in conveying CE particles 134 to jet mill 136.

For example, system 132 includes a splitter 152 downstream from compressor 148 for splitting compressed gas 144 into a first portion 154 and a second portion 156. First portion 154 is provided to jet mill 136, and second portion 156 is provided to nozzle 142 of the feed system, as described above.

In the illustrated embodiment, a pressure regulator 158 is positioned between splitter 152 and jet mill 136. Pressure regulator 158 regulates the pressure and/or velocity of first portion 154 of compressed gas 144 to produce milling gas 160 to be supplied to jet mill 136. This pressure regulation may be based on a desired particle size distribution of CE microparticles 112 produced from CE particles 134 by jet mill 136. In one embodiment or in combination with any embodiment mentioned herein, milling gas 160 (i.e., regulated first portion 154) is supplied to jet mill 136 at a greater velocity than second portion 156 supplied to nozzle 142.

In an alternative embodiment, the nozzle 142 is placed between the regulator 158 and the jet mill 136. The milling gas 160 would feed directly into the nozzle 142 to feed the particle stream 146 into the jet mill. In this set up, there would be no second portion 156 feeding into the nozzle 142.

In one embodiment or in combination with any embodiment mentioned herein, jet mill 136 is a high velocity fluid energy impact mill that circulates CE particles 134 entrained in milling gas 160 therein. Jet mill 136 generally does not include any moving parts. Thus, CE particles 134 are size-reduced by way of facilitating collisions between the individual particles and/or between the particles and portions of jet mill 136 itself.

As described above, the particle size distribution of the resulting CE microparticles 112 produced in jet mill 136 is controlled based on the pressure and/or velocity of milling gas 160 supplied to jet mill 136. In one embodiment or in combination with any embodiment mentioned herein, the milling performed in jet mill 136 reduces the D50 particle size of initial CE particles 134 by at least 25, 50, 75, 85, 90, or 95 percent.

A milled CE stream 164, including CE microparticles 112 entrained in milling gas 160, is provided to a separator 166 from jet mill 136. Separator 166 separates CE microparticles 112 from milling gas 160 to thereby form a CE microparticle stream 168 and a separated gas stream 150. In one embodiment or in combination with any embodiment mentioned herein, the separating is performed by filtering CE microparticles 112 from milling gas 160.

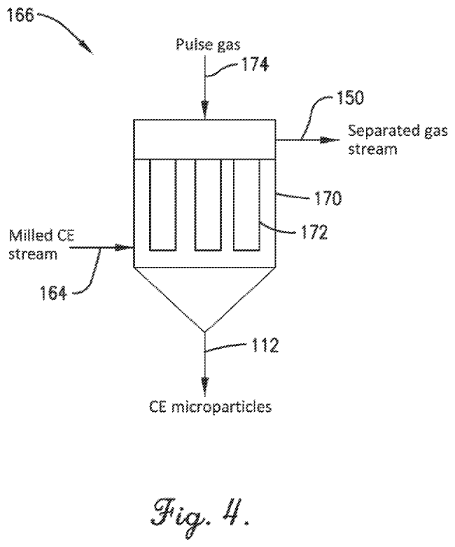

For example, referring to FIG. 4, separator 166 includes a housing 170 and a filter 172 positioned therein. Housing 170 receives milled CE stream 164, and filter 172 separates CE microparticles 112 from milling gas 160. Accordingly, CE microparticles 112 and separated gas stream 150, formed from milling gas 160, are discharged from housing 170 in separate streams. In addition, pulse gas 174 may be provided to housing 170 to dislodge any particles collected on filter 172.

Referring again to FIG. 2, separated gas stream 150 may be recycled/recirculated for use in at least one of jet mill 136, or in conveying the initial CE particles to jet mill 136 via nozzle 142, as described above. In one embodiment or in combination with any embodiment mentioned herein, the recycling is performed by compressing separated gas stream 150 in compressor 148, thereby producing compressed gas 144.

Microparticle CE product stream 168 may be routed to at least one product packaging station 176 for collecting CE microparticles 112. Referring to FIG. 2, microparticle CE product stream 168 is routed to a single product packaging station 176 that collects all the CE microparticles 112 contained therein regardless of relative particle size.

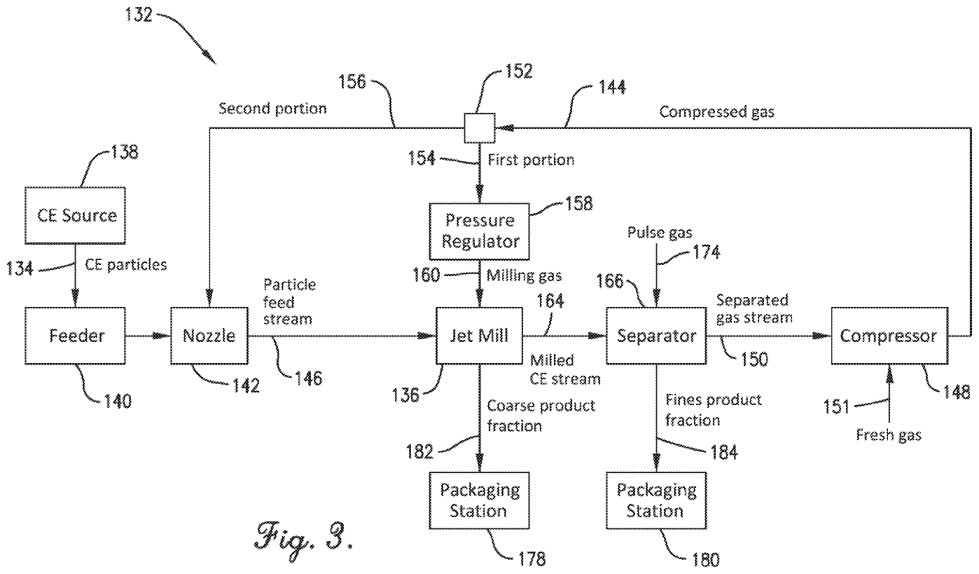

Referring to FIG. 3, CE microparticles are collected at separate product packaging stations, such as a first product packaging station 178 and a second product packaging station 180. In one embodiment or in combination with any embodiment mentioned herein, the rotational circulation of CE particles 134 within jet mill 136 can produce CE microparticle fractions of different sizes at different radial regions within jet mill 136.

In one embodiment or in combination with any embodiment mentioned herein, first product packaging station 178 is for collecting a coarse product fraction 182 of the CE microparticles, and second product packaging station 180 is for collecting a fines product fraction 184 of the CE microparticles. In the illustrated embodiment, first product packaging station 178 receives coarse product fraction 182 directly from jet mill 136. Second product packaging station 180 receives fines product fraction 184 indirectly from jet mill 136. That is, separator 166 is positioned between jet mill 136 and second product packaging station 180.

### Initial CE Particles

As described above, the initial CE particles used to produce the CE microparticles may be derived from a CE production facility, such as that illustrated in FIG. 1, and/or received in bulk packages from a CE manufacturer.

In one embodiment or in combination with any embodiment mentioned herein, the initial CE particles have an average particle size of at least 75 microns.

In one embodiment or in combination with any embodiment mentioned herein, the initial CE particles are in the form of at least one of a powder, flakes, or pellets.

In one embodiment or in combination with any embodiment mentioned herein, the initial CE particles are in the form of a powder having an average particle size of at least 50, 75, 100, 150, 200, 250, 300, 350 or 400 microns and/or not more than 1500, 1250, 1000, 900, 800, 700, 600, or 500 microns.

In one embodiment or in combination with any embodiment mentioned herein, the initial CE particles are in the form of flakes having an average particle size of at least 500, 1000, 2000, 3000, 4000, or 5000 microns and/or not more than 10000, 8000, 6000, 4000, or 2000 microns.

In one embodiment or in combination with any embodiment mentioned herein, the initial CE particles are in the form of pellets having an average particle size of at least 500, 1000, 1500, 2000, 2500, or 3000 microns and/or not more than 20000, 15000, 10000, 8000, 6000, 5000, 4000, or 3000 microns.

The average particle size of the initial CE particles (e.g., powder, flake, or pellets) is determined by measuring the maximum external dimension of 30 randomly selected representative particles and calculating the average of those 30 maximum external dimension values. Measurement of the maximum external dimension is carried out by capturing a two-dimensional image of at least 30 representative particles whose maximum external dimensions are clearly visible. The maximum external dimension of the 30 particles in the two-dimensional image are measured electronically or manually using a caliper-type measurement technique.

In one embodiment or in combination with any embodiment mentioned herein, the initial CE particles comprise CE in an amount of at least 50, 60, 70, 80, 90, 95, or 99 weight percent.

In one embodiment or in combination with any embodiment mentioned herein, the initial CE particles have a moisture content within a range of 0.5-10, 0.5-8.0, 0.5-6.0, or 0.5-5.0 weight percent.

### CE Microparticles

The CE microparticles produced by the processes disclosed herein exhibit enhanced solidity and/or tactile feel, for example, making them desirable for use in personal care products, cosmetics, and the like.

In one embodiment or in combination with any embodiment mentioned herein, the CE microparticles have a D50 particle size in the range of 0.5 to 100, 1 to 100, 1 to 80, 1 to 70, 1 to 60, 1 to 50, 1 to 40, 1 to 35, 1 to 30, 1 to 25, 1 to 20, 1 to 15, 1 to 10, 2 to 100, 2 to 80, 2 to 70, 2 to 60, 2 to 50, 2 to 40, 2 to 35, 2 to 30, 2 to 25, 2 to 20, 2 to 15, 2 to 10, 3 to 100, 3 to 80, 3 to 70, 3 to 60, 3 to 50, 3 to 40, 3 to 35, 3 to 30, 3 to 25, 3 to 20, 3 to 15, 3 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the hardened CE microparticles can have a D50 particle size of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 microns.

As used herein, the term “D50” means that 50% of the microparticles have a maximum dimension that is less than or equal to the noted value (e.g., 10 microns), based on a volume basis. The D50 value may also be treated as the median particle size. To ensure that a representative D50 value is obtained, the sample size of the microparticles should be at least 0.5 grams. The microparticle sample size is then dispersed and mixed in 1.5 ounces of isopropanol or aqueous surfactant solution (1 drop 5% v/v Igepal® CO-630 surfactant). Testing for D50 is performed via laser diffraction and computer algorithms using the Mie theory to generate a particle size distribution. One suitable particle size analyzer for determining D50 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D10 particle size of 0.5 to 20, 0.5 to 15, 0.5 to 12, 0.5 to 10, 0.5 to 5, 0.5 to 4, 0.5 to 3, 0.5 to 2, 0.5 to 1, 1 to 20, 1 to 15, 1 to 12, 1 to 5, 1 to 3, 2 to 20, 2 to 10, 2 to 5, 3 to 20, 3 to 15, 3 to 10, 4 to 20, 4 to 15, 4 to 10, 5 to 20, 5 to 15, 5 to 10, 10 to 20, or 10 to 15 microns. For example, the CE microparticles can have a D10 particle size of 0.5, 1, 2, 3, 4, 5, 6, 7, 8, 9, 10, 11, 12, 13, 14, 15, 16, 17, 18, 19, or 20 microns.

As used herein, the term “D10” means that 10% of the microparticles have a maximum dimension that is less than or equal to the noted value (e.g., 10 microns), based on a volume basis. To ensure that a representative D10 value is obtained, the sample size of the microparticles should be at least 0.5 grams. The microparticle sample size is then dispersed and mixed in 1.5 ounces of isopropanol or aqueous surfactant solution (1 drop 5% v/v Igepal®) CO-630 surfactant). Testing for D10 is performed via laser diffraction and computer algorithms using the Mie theory to generate a particle size distribution. One suitable particle size analyzer for determining D10 values is the Malvern Mastersizer 3000 from Malvern Panalytical. When using the

Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D90 particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D90 particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D90” means that 90% of the microparticles have a maximum dimension that is less than or equal to the noted value (e.g., 10 microns), based on a volume basis. To ensure that a representative D90 value is obtained, the sample size of the microparticles should be at least 0.5 grams. The microparticle sample size is then dispersed and mixed in 1.5 ounces of isopropanol or aqueous surfactant solution (1 drop 5% v/v Igepal® CO-630 surfactant). Testing for D90 is performed via laser diffraction and computer algorithms using the Mie theory to generate a particle size distribution. One suitable particle size analyzer for determining D90 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D100 particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D100 particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D100” means that 100% of the microparticles have a maximum dimension that is less than or equal to the noted value (e.g., 10 microns), based on a volume basis. To ensure that a representative D100 value is obtained, the sample size of the microparticles should be at least 0.5 grams. The microparticle sample size is then dispersed and mixed in 1.5 ounces of isopropanol or aqueous surfactant solution (1 drop 5% v/v Igepal® CO-630 surfactant). Testing for D100 is performed via laser diffraction and computer algorithms using the Mie theory to generate a particle size distribution. One suitable particle size analyzer for determining D100 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles exhibit at least 50, 55, 60, 65, 70, 75, 80, or 85 percent biodegradability at 60 days according to at least one of the OECD 301B, OECD 301C, OECD 301D, OECD 301F, OECD TG 310, OECD TG 306, ISO 14852, or ISO 14851 test methods.

In one embodiment or in combination with any embodiment mentioned herein, the CE microparticles exhibit an oil absorption rate of at least 0.5, 0.6, 0.7, or 0.8 g/g according to ASTM D281 using olive oil instead of castor oil.

The oil absorption rate was determined in accordance with a modification of the ASTM D281 standard test method, which is used to determine the oil absorption of micropowders. In the modified method, a known amount of micropowder (˜0.20 g) was weighed in a glass vial and olive oil was added drop-by-drop to the micropowder with a pipette. The powder was thoroughly mixed with oil after the addition of every other droplet of olive oil. The test was completed when the amount of oil incorporated with the powder to produce a very stiff, putty-like paste which did not break and separate. The dropping bottle containing oil was then weighed to determine the amount of oil added to the powder.

The oil absorption capability was calculated in accordance with the following equation:

$$Oil Absorption ( g / g ) = A - B / W ,$$

wherein A=the initial weight of the dropping bottle with oil, B=the final weight of the dropping bottle with oil, and W=the weight of the sample in grams. The experiment was performed under the same conditions and repeated at least two additional times. The oil absorption rate was determined from an average of the results of the experiments.

In one embodiment or in combination with any embodiment mentioned herein, the CE microparticles exhibit a monomodal particle size distribution with a span of at least 0.5, 0.55, 0.6, 0.65, 0.7, 0.75, 0.8, 0.85, 0.9, 0.95, 1.0, 1.05, 1.1, 1.15, 1.2, 1.25, 1.3, or 1.4 and/or less than 3.0, 2.9, 2.8, 2.7, 2.6, 2.5, 2.4, 2.3, 2.2, 2.1, 2.0, 1.9, 1.8, 1.7, 1.6, or 1.5. In certain embodiments, the CE microparticles exhibit a monomodal particle size distribution with a span of 1.0 to 3.0, 1.0 to 2.5, 1.0 to 2.0, 1.0 to 1.8, 1.0 to 1.6, 1.2 to 3.0, 1.2 to 2.5, 1.2 to 2.0, 1.2 to 1.8, 1.2 to 1.6, 1.3 to 3.0, 1.3 to 2.5, 1.3 to 2.0, 1.3 to 1.8, or 1.3 to 1.6. As used herein, “monomodal particle size distribution” refers to a particle size distribution for a material that only has a single notable peak of size distribution. This is in contrast to multi-modal particle size distributions, which will have two or more peaks of particle size distributions. The “span” of the monomodal peak may be measured using the D10, D50, and D90 values of the particles using the following formula:

$$( {D}_{x} ( 90 ) - {D}_{x} ( 10 ) ) / {D}_{x} ( 50 ) ,$$

wherein “x” is the designated particle size.

In one embodiment or in combination with any embodiment mentioned herein, the CE microparticles have a polydispersity index of less than 0.8, 0.7, 0.6, 0.5, 0.4, or 0.3 and a sphericity of less than 70, 65, 60, 55, 50, 45, 40, 35, or 30 percent.

Average sphericity is determined by: (1) obtaining a secondary emission/ETD detector scanning electron microscopy (SEM) image of a representative sample of at least 40 microparticles, (2) on the SEM image, selecting a square sample window centered at the center of the SEM that contains exactly 30 microparticles whose entire outer perimeters are clearly visible (i.e., not occluded), (3) measuring the maximum and minimum diameters (each extending through the particle's centroid and not necessarily perpendicular to one another) of the 30 clearly visible microparticles in the sample window, (4) for each of the 30 particles, dividing the minimum diameter by the maximum diameter and multiplying the result by 100% to obtain 30 individual particle sphericities, and (5) averaging the 30 individual particle sphericities to obtain the average sphericity.

In one embodiment or in combination with any embodiment mentioned herein, the CE microparticles have a butyric acid content of less than 500, 400, 300, 200, 100, 50, 20, 10, 7.5, 5, 2.5, or 1 ppmw.

In one embodiment or in combination with any embodiment mentioned herein, the CE microparticles have an acetic acid content of less than 500, 400, 300, 200, 100, 50, 20, 10, 7.5, 5, 2.5, or 1 ppmw.

In one embodiment or in combination with any embodiment mentioned herein, the CE microparticles have a propionic acid content of less than 500, 400, 300, 200, 100, 50, 20, 10, 7.5, 5, 2.5, or 1 ppmw.

The butyric acid, acetic acid, and propionic acid contents of the CE microparticles may be measured via gas chromatography (“GC”). Under one GC methodology, the butyric acid, acetic acid, and propionic acid contents may be measured by adding about 100 mg of the CE microparticles to a tared 4-dram vial, followed by the addition of an internal standard solution comprising nonane in a 90:10 mixture of dichloromethane/methanol. A magnetic stir bar is placed in the vial, and the sample is stirred for two hours. After stirring, 8.0 mL of n-heptane is added dropwise to precipitate the polymer, and then the sample is vortexed. Approximately 100 mg of the supernatant is transferred to a GC vial, along with 100 μL of pyridine and 450 μL of BSTFA. The samples are heated at 80° C. for 30 minutes and then cooled to room temperature before injection. Samples are chromatographed simultaneously on 100% dimethylpolysiloxane and 14% cyanopropyl-phenyl-methylpolysiloxane columns using temperature programming and flame ionization detection. Alternatively, a second GC methodology involves preparing samples by adding approximately 30 mg of CE microparticles to a tared GC vial, followed by 200 μL of an internal standard solution comprising decane in pyridine, and 1.0 mL of BSTFA. The vials re heated at 80° C. for 30 minutes and then cooled to room temperature before injection. Samples are then chromatographed simultaneously on 100% dimethylpolysiloxane and 6% cyanopropyl-phenyl-methylpolysiloxane columns using temperature programming and flame ionization detection.

In one embodiment or in combination with any embodiment mentioned herein, the CE microparticles have a sulfuric acid content of less than 500, 400, 300, 200, 100, 50, 20, 10, 7.5, 5, 2.5, or 1 ppmw. The sulfuric acid content of the CE microparticles may be measured by the following methodology. First, the tested sample is added to a titration cell and dissolved in a solvent for a total volume of 70 mL. Solvent blanks are also prepared for comparison purposes. The samples and blanks are then titrated with 0.05 N potassium hydroxide in methanol using an automatic titrator equipped with a combination glass potentiometric electrode. Acid number is calculated based on the sample weight and the KOH consumed in the sample minus the KOH consumed in the blank.

In one embodiment or in combination with any embodiment mentioned herein, the CE microparticles have a CE content of at least 50, 55, 60, 65, 70, 75, 80, 85, 90, 91, 92, 93, 94, 95, 96, 97, 98, or 99 weight percent of the mixed cellulose ester of the first aspect, the second aspect, and/or the third aspect, including any class or subclass of these aspects. Additionally, or in the alternative, the CE microparticles may have a CE content of less than 99.9, 99.5, 99, 98, 97, 96, 95, 94, 93, 92, 91, 90, 89, 88, 87, 86, or 85 weight percent of the mixed cellulose ester of the first aspect, the second aspect, and/or the third aspect, including any class or subclass of these aspects. In certain embodiments, the CE microparticles may consist essentially of the mixed cellulose ester of the first aspect, the second aspect, and/or the third aspect, including any class or subclass of these aspects.

In one embodiment or in combination with any embodiment mentioned herein, the CE microparticles may contain an additional biodegradable cellulose ester that is different from the mixed cellulose ester of the first aspect, the second aspect, and/or the third aspect. In such embodiments, this additional cellulose ester can be cellulose acetate, which exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, OECD 301D, OECD 301F, OECD TG 310, OECD TG 306, ISO 14852, or ISO 14851 test methods.

In one embodiment or in combination with any embodiment mentioned herein, the CE microparticles may contain at least 1, 2, 3, 4, 5, 6, 7, 8, 9, or 10 weight percent the additional biodegradable cellulose ester that is different from the mixed cellulose ester of the first aspect, the second aspect, and/or the third aspect. Additionally, or in the alternative, the CE microparticles may contain less than 50, 45, 40, 35, 30, 25, 20, 15, 10, or 5 weight percent of the additional biodegradable cellulose ester that is different from the mixed cellulose ester of the first aspect, the second aspect, and/or the third aspect.

In one embodiment or in combination with any embodiment mentioned herein, the CE microparticles have an average BET surface area of at least 0.1, 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4. 6.5, 6.7, 6.8, 6.9, 7.0, 7.1, 7.2, 7.3, 7.4, 7.5, 7.6, 7.7, 7.8, 7.9, or 8.0 m²/g and/or not more than 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.9, 1.8, 1.7, 1.6, 1.5, 1.4, or 1.3 m²/g as measured according to ISO 9277 using a Micromeritics ASAP 2020 instrument and krypton gas. The measurement method includes the following steps: (1) 0.5-1 gram samples are degassed at 60° C. overnight; (2) Sample mass is collected by the weight difference of the empty sample tube and the sample tube filled with sample after degassing; (3) Krypton adsorption at 77K is used for the specific surface area analysis; and (4) Seven relative pressures from 0.06 to 0.20 are collected and fitted for BET specific surface area analysis. The amount of sample is dependent on specific need and the level of moisture in them.

In one embodiment or in combination with any embodiment mentioned herein, the CE microparticles have bulk density in the range of 0.2 to 0.3 g/m³

In one embodiment or in combination with any embodiment mentioned herein, the CE microparticles have an average sphericity of at least at least 20, 30, 40, 50, or 60 percent and/or not more than 90, 80, 70, 60, or 50 percent.

In one embodiment or in combination with any embodiment mentioned herein, the CE microparticles have an average smoothness of at least 10, 20, 30, 40, 50, 60, 70, 80, 90, 95, 95, 97, 98, or 99 percent and/or not more than 99, 95, 90, 80, 70, 60, 50, 40, 30, 20, or 10 percent.

Average smoothness is determined by: (1) obtaining a secondary emission/EDT detector scanning electron microscopy (SEM) image of a representative sample of at least 20 microparticles, (2) on the SEM, selecting a square sample window centered at the center of the SEM that contains exactly 10 microparticles whose entire outer perimeters are clearly visible (i.e., not occluded), (3) binarizing the sample window by manual binarization with upper and lower thresholds chosen to match the exact shape of the darker regions of the particles, (4) for each of the 10 microparticles, selecting a square window at or near the center of the particle having length and width that are approximately ⅓ of the particle diameter (before binarizing the particle), (5) dividing the dark region area in the square window by the total area of the square window and multiplying the result by 100% to obtain 10 individual particle smoothnesses, and (5) averaging the 10 individual particle smoothnesses to obtain the average smoothness.

In one embodiment or in combination with any embodiment mentioned herein, the CE microparticles have a moisture content that is at least 0.1, 0.25, 0.5, 1, 2, 4, or 5 weight percent less than the moisture content of the initial CE particles. For example, it is believed milling the initial CE particles in the presence of milling gas, such as in jet mill 136, reduces the moisture content of the CE microparticles relative to the initial CE particles.

In one embodiment or in combination with any embodiment mentioned herein, the CE microparticles have a cumulative amount of surfactants of less than 100 ppmw, 75 ppmw, 50 ppmw, 25 ppmw, 10 ppmw, or 5 ppmw.

In one embodiment or in combination with any embodiment mentioned herein, the CE microparticles have a cumulative amount of hydrocolloids of less than 100 ppmw, 75 ppmw, 50 ppmw, 25 ppmw, 10 ppmw, or 5 ppmw.

In one embodiment or in combination with any embodiment mentioned herein, the CE microparticles have a cumulative amount of water-soluble polymers of less than 100 ppmw, 75 ppmw, 50 ppmw, 25 ppmw, 10 ppmw, or 5 ppmw. As used herein, a “water-soluble polymer” refers to a polymer having an insoluble content of less than 50 wt. % when 1 g of the polymer is dissolved in 100 g of water at 25° C.

In one embodiment or in combination with any embodiment mentioned herein, the CE microparticles have a cumulative amount of surfactants, hydrocolloids, and water-soluble polymers of less than 100 ppmw, 75 ppmw, 50 ppmw, 25 ppmw, 10 ppmw, or 5 ppmw.

In one embodiment or in combination with any embodiment mentioned herein, the CE microparticles have a true specific gravity of at least 0.3, 0.4, 0.5, 0.6, 0.7, 0.8, or 0.9 and/or not more than 1.5, 1.4, 1.3, 1.2, 1.1, 1.0, 0.9, 0.8, 0.7, or 0.6 as measured by JIS Z8807-1976.

In one embodiment or in combination with any embodiment mentioned herein, the CE microparticles have a bulk specific gravity of at least 0.2, 0.3, 0.4, 0.5, 0.6, 0.7, or 0.8 and/or not more than 1.4, 1.3, 1.2, 1.1, 1.0, 0.9, 0.8, 0.7, 0.6, or 0.5 as measured by JIS 1201-1.

### Cosmetic Formulations

The CE microparticles produced by the processes disclosed herein may be used to produce a variety of cosmetic compositions. The cosmetic compositions may be produced by: (1) providing a plurality of the CE microparticles; (2) combining the CE microparticles with one or more cosmetic additives to thereby form a pre-cosmetic mixture; and (3) forming the cosmetic composition from the pre-cosmetic mixture.

In one embodiment or in combination with any other embodiment mentioned herein, the cosmetic composition can comprise at least 0.1, 0.5, 1, 2, 3, 4, 5, 6, 7, 8, 9, 10, 11, 12, 13, 14, or 15 weight percent of the CE microparticles. Additionally, or in the alternative, the cosmetic composition can comprise less than 99, 90, 80, 70, 60, 50, 40, 30, 25, 20, 15, 10, or 5 weight percent of the CE microparticles. For example, the cosmetic composition can comprise 0.1 to 90, 0.1 to 50, 0.1 to 30, 0.1 to 20, 0.1 to 15, 0.1 to 10, 0.1 to 5, 1 to 90, 1 to 50, 1 to 30, 1 to 20, 1 to 15, 1 to 10, or 1 to 5 weight percent of the CE microparticles.

In one embodiment or in combination with any other embodiment mentioned herein, the cosmetic composition can be a foundation, a sunscreen, a lipstick, a mascara, an eye shadow, a lotion, a dry shampoo, a liquid shampoo, a body wash, a lotion, a gas conditioner, a skin moisturizer, a face wash, a tablet, a foot powder, a baby powder, a shaving cream, or a shaving gel.

In one embodiment or in combination with any other embodiment mentioned herein, the cosmetic composition can be a loose powder, a compacted powder, a gel, an emulsion, a liquid, or an aerosol.

In one embodiment or in combination with any other embodiment mentioned herein, the cosmetic composition comprises at least 1, 5, 10, 15, 20, 25, 30, 35, 40, 45, 50, 55, 60, 65, 70, 75, 80, 85, 90, 95, or 99 weight percent of at least one, two, three, four, or five cosmetic additives.

Additionally, or in the alternative, the cosmetic composition can comprise less than 99, 95, 90, 85, 80, 75, 70, 65, 60, 55, or 50 weight percent of at least one, two, three, four, or five cosmetic additives. For example, the cosmetic composition can comprise 1 to 99, 1 to 95, 1 to 90, 1 to 85, 1 to 80, 5 to 99, 5 to 95, 5 to 90, 5 to 85, 10 to 99, 10 to 95, 10 to 85, 10 to 80, 15 to 99, 15 to 95, 15 to 90, 15 to 85, or 15 to 80 weight percent of at least one, two, three, four, or five cosmetic additives.

Generally, the cosmetic additives can include a solvent, a colorant, an oil, a wax, a fatty acid, an alcohol, an ester, a hydrocarbon, a silicone oil, a surfactant, a metal soap, a moisturizer, a thickener, a UV absorber, an antioxidant, an oil absorbent, an exfoliant, water, or a combination thereof.

In one embodiment or in combination with any other embodiment mentioned herein, the colorant comprises a pigment (e.g., an organic pigment and/or an inorganic pigment) and/or a dye.

In one embodiment or in combination with any other embodiment mentioned herein, the oil comprises triglycine, soybean oil, cocoa butter, palm oil, palm kernel oil, hardened oil, and/or hardened castor oil.

In one embodiment or in combination with any other embodiment mentioned herein, the wax comprises carnauba wax, candelilla wax, lanolin, lanolin, candelilla wax, cotton wax, Montan wax, Kapok wax, lanolin acetate, lanolin, and/or lanolin fatty acid isopropyl.

In one embodiment or in combination with any other embodiment mentioned herein, the fatty acid comprises lauric acid, myristic acid, palmitic acid, stearic acid, isostearic acid, behenic acid, oleic acid, undecylenic acid, linoleic acid, eicosapentaenoic acid (EPA), and/or docosahexaenoic acid.

In one embodiment or in combination with any other embodiment mentioned herein, the alcohol comprises cetyl alcohol, stearyl alcohol, isostearyl alcohol, 2-octyldodecanol, lauryl alcohol, behenyl alcohol, myristyl alcohol, oleyl alcohol, and/or cetostearyl alcohol.

In one embodiment or in combination with any other embodiment mentioned herein, the ester comprises isopropyl myristate, 2-octyldodecyl myristate, cetyl 2-ethylhexanoate, diisostearyl malate, tripropylene glycol dineopentate, isononyl isononanoate, isotorideyl isononanoate, cetyl octanoate, isocetyl palmitate, butyl stearate, hexyl laurate, myristyl myristate, decyl oleate, hexyl decyl dimethyloctanate, cetyl lactate, myristyl lactate, lanolin acetate, isosetyl stearate, isosetyl isostearate, cholesteryl 12-hydroxystearate, di-2-ethylhexanoic acid ethylene glycol, dipentaerythritol fatty acid ester, monoisostearate N-alkylglycol, dicaprate neopentyl glycol, di-2-heptylundecanoate glycerin, tri-2-ethylhexanoate trimethylpropane, Trimethylolpropane triisostearate, pentaerythritol tetra-2-ethylhexanoate, glycerin tri-2-ethylhexanoate, glycerin trioctanoate, glycerin triisopalmitate, trimethylolpropane triisostearate, ethylhexyl palmitate, glycerin trimyristate, tri-2-heptylundecanoic acid glyceride, castor oil fatty acid methyl ester, oleyl oleate, acetoglyceride, 2-heptylundecyl palmitate, diisobutyl adipate, N-lauroyl-L-Glutamic hexyldecyl palmitate, adipate hexyldecyl, diisopropyl sebacate, ethylhexyl succinate, and/or triethyl citrate.

In one embodiment or in combination with any other embodiment mentioned herein, the hydrocarbon comprises paraffin, petrolatum, and/or microcrystalline wax.

In one embodiment or in combination with any other embodiment mentioned herein, the surfactant comprises an anionic surfactant, a cationic surfactant, and/or a nonionic surfactant.

In one embodiment or in combination with any other embodiment mentioned herein, the thickener comprises guar gum, pectin, starch, gelatin, collagen, cellulosic derivatives, and/or mannan.

### Experiments

### Abbreviations

Ex is example; DS is average degree of substitution; DS_{OH} is average degree of substitution for hydroxyl; DS_{Ac} is the average degree of substitution for acetyl; DS_{Bu} is the average degree of substitution for butyryl; M_{w} is weight average molecular weight; Pr₂O is propionic anhydride; PrOH is propionic acid; g is gram(s), ° C. is degrees Celsius; Ac₂O is acetic anhydride; min is minute(s); AcOH is acetic acid; Mg(OAc)₂ is magnesium acetate; OAc is acetate; rt is room temperature; soln is solution; kg is kilogram(s).

Preparation of Cellulose Acetate Butyrate (1) (DS_{Ac}=2.23, DS_{Bu}=0.22, DS_{OH}=0.55 M_{w}=50,000-90,000)

Cellulose and acid mixture [cellulose (5.1 parts) and AcOH (20.6 parts)] was added to an agitated reactor and soaked unheated then, the mixture was heated to 55° C. Some amount of sulfuric acid was added and the reactor was cooled to 30° C. Following, a mixture of Ac₂O (11 parts) and Bu₂O (7.1parts) was added and the mixture cooled to around 9° C. with agitation. Additional sulfuric acid was added to a total of 0.8 parts and the resulting reaction mixture was warmed to 50° C. until the acylation was complete and the desired molecular weight is achieved. To this reaction mixture was added a mixture of AcOH (23.3 parts) and H₂O (9.1 parts). The mixture was then stirred at 68° C. for 720 min, but with the addition of a mixture Mg(OAc)₂ (0.77 parts), BuOH (8.7 parts) and H₂O (3.4 parts) after 80 min. After the full length of time, the mixture was fully neutralized with a solution of Mg(OAc)₂ (1.1 parts), BuOH (1.9 parts) and H₂O (6.1 parts). The mixture was then precipitated in water, washed and dried by common methods.

### Degree of Substitution

The degree of substitution for the substituents on the cellulose ester backbone is calculated using proton nuclear magnetic resonance spectroscopy. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

### Molecular Weight

The molecular weight is determined by gel permeation chromatography. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

By adapting the procedure for the preparation of cellulose acetate butyrate 1, the following cellulose ester in Table 1 were prepared.

TABLE 1
Preparation conditions for Ex 2-5.

| | Activated Cellulose | | | | | Hydrolysis | | Hydrolysis | |
|---|---|---|---|---|---|---|---|---|---|
| | Mixture | | Acylation Solution | | | Soln | | Conditions | |
| | Cellulose | Acid | H₂SO₄ | Ac₂O | Bu₂O | BuOH | H₂O | Time | Temp. |
| Ex | (parts) | (parts) | (parts) | (parts) | (parts) | (parts) | (parts) | (min) | (° C.) |
| 2 | 5.1 | AcOH (20.6) | 0.8 | 11 | 7.1 | 10.6 | 9.5 | 840 | 68 |
| 3 | 5.1 | AcOH (20.6) | 0.8 | 11 | 7.1 | 10.6 | 9.5 | 720 | 68 |
| 4 | 5.1 | AcOH (20.6) | 0.8 | 11 | 7.1 | 10.6 | 9.5 | 720 | 68 |
| 5 | 5.1 | AcOH (20.6) | 0.8 | 11 | 7.1 | 10.6 | 9.5 | 1020 | 68 |

Table 2 provides properties for the cellulose esters prepared above.

TABLE 2
Degrees of substitution and molecular weight for Ex 2-5.

| Ex | DS_{Ac} | DS_{Bu} | Total DS | DS_{OH} | M_{w} (Da) |
|---|---|---|---|---|---|
| 2 | 1.99 | 0.2 | 2.19 | 0.81 | 72208 |
| 3 | 2.23 | 0.22 | 1.96 | 0.55 | 70266 |
| 4 | 2.08 | 0.21 | 2.24 | 0.7 | 68278 |
| 5 | 1.86 | 0.2 | 2.14 | 0.92 | 78174 |

### Jet Milling

There are multiple jet-milling configurations that can be used to reduce the size of particles. Such configurations are discussed in A. Chamayou and J. A. Dodds, Air Jet Milling, Handbook of Powder Technology, volume 12, Chapter 8, 2007 (“Chamayou”). FIG. 7 of Chamayou provides an example of a fluidized bed opposed jet mill that can be used to reduce the size of the cellulose ester particles described below in Table 3. The jet-milling process was used to reduce cellulose ester average particle size from 300-900 μm to ˜10 μm. Fluidized bed opposed jet mills operates as follows: The cellulose ester is placed into a hopper and introduced into the top of the mill (“FEED IN”) typically though a double valve arrangement (or through an injector). The cellulose ester particles fall by gravity to the bottom of the mill where they are swept up into one of three high pressure air streams that are geometrically oriented towards one another thus forming the so-called “pulverizing zone.” Within the pulverizing zone, the cellulose ester particles are size reduced via interparticle collisions. The size-reduced particles are then conveyed upwards by mass transport in the vertical airstream (fluidized bed) ultimately carrying them into the classifier. The classifier allows particles below the desired minimum size to be removed from the mill (“FINE OUT”). Particles that are above the maximum size are excluded from the classifier and returned to the fluidized bed eventually falling back down into the pulverizing zone for further size reduction. Particles that fall within the desired size range are ejected from the classifier into an appropriate product container. Many control parameters exist for optimizing productivity, particle size and particle size distribution shape, including but not necessarily limited to, classifier rotor speed, air nozzle pressure, and bed level.

### Moisture Determination

The moisture levels were determined using 1 gram of material with a Torbal Moisture balance with the material being heated to 115° F.

### Refractive Index Determination

The refractive indexes of the cellulose esters were determined by preparing films of the cellulose esters using a 12 wt % solids in a solution of either methylene chloride/methanol (9:1) or methylene chloride/ethanol solutions (9:1) to make a dope. The films were cast from the dope onto a glass substrate and evenly spread onto the glass substrate using a casting knife. The wet dope on the glass substrate was covered and the solvent was allowed to slowly evaporate for 45 minutes while covered. The films were then allowed to evaporate for 15 minutes without the cover. The films were peeled from the substrate and put between two papers and set aside using a weight to ensure that the films were flat (˜2 hours). The films were then put between metal frames and allowed to anneal for 10 minutes in an oven set at 100° C. and 10 minutes in another oven set at 120° C. The resulting films are used to determine the refractive index using a Metricon (Prism coupler method) using a wavelength of 589 nm.

### Young's Modulus

Films were prepared as is disclosed in the procedure for determining the refractive index. The films were cut into 6 inch by 0.5 inch specimens. The Young's modulus is determined by testing 5 specimens. Each specimen is tested in tensile mode at 0.2 inches/minute up to 1.6% strain and then at 2 inches/minute until the film breaks.

### OECD 301F Test Description

In this experiment, a measured volume of a mineral medium is inoculated with a known concentration of the test substance, which serves as nominal source of organic carbon. The medium is placed in a closed flask and stirred at a constant temperature (within a range of +1° C. or closer) for a maximum of 60 days.

The consumption of oxygen is determined using one of two methods: either by measuring the amount of oxygen required (produced electrolytically) to maintain a constant gas volume in the respirometer flask, or by monitoring changes in volume or pressure (or a combination of both) in the apparatus.

Any carbon dioxide produced during the process is absorbed using a solution of potassium hydroxide or another suitable absorbent. The amount of oxygen utilized by the microbial population during the biodegradation of the test substance is calculated by subtracting the oxygen uptake by the blank inoculum (which runs in parallel). This value is expressed as a percentage of Theoretical Oxygen Demand (ThOD) or, less ideally, Chemical Oxygen Demand (COD)

TABLE 3

| | | | | Particle | Refractive | Bulk | | Oil | Youngs |
|---|---|---|---|---|---|---|---|---|---|
| | CE | Microbeads | | Size | Index | Density | Moisture | Absorp. | Modulus |
| Sample | (Ex #) | Process | DS_{OH} | (μ) | (589 nm) | (g/ml) | (wt. %) | (g/g) | (GPa) |
| 1 | 1 | Jet Mill | 0.6 | 11.3 | 1.48 | 0.20 | 3.0 | 1.23 | — |
| 2 | 1 | Jet Mill | 0.6 | 11.3 | — | 0.22 | 3.7 | — | — |
| 3 | 1 | Jet Mill | 0.6 | 11.6 | — | 0.22 | 3.0 | — | — |
| 4 | 1 | Jet Mill | 0.6 | 11.6 | — | 0.22 | 2.4 | — | — |
| 5 | 1 | Jet Mill | 0.6 | 10.2 | — | — | — | 1.16 | — |
| 6 | 2 | Jet Mill | 0.8 | 7.03 | — | 0.298 | 2.5 | — | — |
| 7 | 3 | Jet Mill | 0.55 | 6.67 | — | — | 0.9 | 0.70 | — |
| 8 | 4 | Jet Mill | 0.69 | 6.57 | — | 0.292 | 2.2 | 0.73 | — |
| 9 | 5 | Jet Mill | 0.92 | 7.01 | — | 0.324 | 2.1 | 0.74 | — |
| Comp. | 2 | Emulsion | — | — | 1.48 | 0.33 | — | 0.69 | 2.1 |
| Sample 10 | | | | | | | | | |
| Nylon 12 | — | — | — | — | 1.52* | 0.398 | — | 0.58 | 1.7-2.2 |
| (Kobo SP-10) | | | | | | | | | |
| PMMA | — | — | — | — | 1.49 | 0.436 | — | 0.46 | 2.3 |
| (Kobo MSP-822) | | | | | | | | | |

Table 4 provides additional CE microparticles made by jet milling. These particles were shown to be greater than 60% biodegradable under the OECD 301F biodegradation test.

TABLE 4

| Sample | | | |
|---|---|---|---|
| # | Microparticle | Material | Description |
| 6-1 | Milled CE | Ex 6 | Prepared by jet mill, Spheroid shape, |
| | Microparticles | | D(10) = 3.85 μm, D(50) = |
| | | | 6.81 μm, D(90) = 11.5 μm, |
| | | | Span = 1.1, Certified >60% |
| | | | biodegradable under OECD 301F |
| 7-1 | Milled CE | Ex 7 | Prepared by jet mill, spheroid shaped, |
| | Microparticles | | Dx(10) = 3.15 μm, Dx(50) = |
| | | | 6.4 μm, Dx(90) = 11.3 μm, |
| | | | Span = 1.3, Certified >60% |
| | | | biodegradable under OECD 301F |
| 8-1 | Milled CE | Ex 8 | Prepared by jet mill, spheroid shape, |
| | Microparticles | | Dx(10) = 3.16 μm, Dx(50) = |
| | | | 6.47 μm, Dx(90) = 11.8 μm, |
| | | | Span = 1.3 |

1. A method of producing biodegradable cellulose ester (CE) microparticles, the method comprising:
(a) providing initial CE particles having an average particle size of at least 75 microns; and
(b) milling the initial CE particles to thereby form CE microparticles having a D50 particle size in the range of 0.5 to 50 microns.

2. The method in accordance with claim 1, wherein the milling of step (b) is performed by jet milling or bead milling the initial CE particles.

3. The method of claim 1, further comprising size-reducing the initial CE particles prior to performing the milling of step (b).

4. The method of claim 3, wherein the size-reducing is performed by at least one of ball milling, rod milling, hammer milling, pin milling, cryogenic milling, or gas classifier milling.

5. The method of claim 1, wherein the milling of step (b) reduces the D50 particle size of the initial CE particles by at least 25, or 50, or 75, or 85, or 90, or 95 percent.

6. The method of claim 1, further comprising providing compressed gas for use in at least one of the milling of step (b), or in conveying the initial CE particles to the milling of step (b).

7. The method of claim 6, further comprising:
supplying a first portion of the compressed gas for use in the milling of step (b); and
supplying a second portion of the compressed gas for use in conveying the initial CE particles to the milling of step (b), wherein the first portion is supplied at a greater velocity than the second portion.

8. The method of claim 1, further comprising separating the CE microparticles, produced from the milling of step (b), from milling gas to thereby form a CE product stream and a separated gas stream.

9. The method of claim 8, further comprising recycling the separated gas stream for use in at least one of the milling of step (b), or in conveying the initial CE particles to the milling of step (b).

10. The method of claim 9, wherein the recycling is performed by compressing the separated gas stream.

11. The method of claim 8, wherein the separating is performed by filtering the CE microparticles from the milling gas.

12. The method of claim 1, further comprising collecting a coarse product fraction and a fines product fraction of the CE microparticles at separate product packaging stations.

13. A system for producing cellulose ester (CE) microparticles, the system comprising:
a source of initial CE particles;
a compressor for producing compressed gas;
a jet mill for receiving and size-reducing the initial CE particles into CE microparticles, wherein the jet mill is supplied with a first portion of the compressed gas from the compressor; and
a feed system for supplying the initial CE particles to the jet mill, wherein the feed system is supplied with a second portion of the compressed gas from the compressor.

14. The system of claim 13, further comprising a separator for receiving the CE microparticles entrained in milling gas from the jet mill, wherein the separator separates the CE microparticles from the milling gas to thereby form a CE product stream and a separated gas stream.

15. The system of claim 14, wherein the separator comprises a filter for separating the CE microparticles from the milling gas.

16. The system of claim 14, wherein the compressor compresses the separated gas stream for provision to at least one of the jet mill or the feed system as at least a portion of the compressed gas.

17. The system of claim 13, further comprising at least one product packaging station for collecting the CE microparticles.

18. The system of claim 13, further comprising a splitter downstream from the compressor for splitting the compressed gas into the first and second portions.

19. The system of claim 13, wherein the feed system comprises a loss-in-weight feeder for controlling a feed rate of the initial CE particles provided to the jet mill.

20. The system of claim 13, wherein the feed system comprises a venturi nozzle for combining the initial CE particles with the second portion of the compressed gas to thereby form a particle feed stream for providing to the jet mill.

21. The system in accordance with claim 13, further comprising a mechanical mill upstream from the jet mill, the mechanical mill for pre-milling the initial CE particles before being provided to the jet mill.

22. The method of claim 1, wherein the initial CE particles are in the form of at least one of a powder, flakes, or pellets.

23. Biodegradable cellulose ester (CE) microparticles comprising at least one CE, wherein the CE microparticles have a D50 particle size in the range of 0.5 to 50 microns, wherein the CE microparticles exhibit at least 50 percent biodegradability at 60 days according to at least one of the OECD 301B, OECD 301C, OECD 301D, OECD 301F, OECD TG 310, OECD TG 306, ISO 14852, or ISO 14851 test methods, wherein the cumulative amount of surfactants, hydrocolloids, and water-soluble polymers present in the CE microparticles is less than 100 ppmw.

24. The method of claim 1, wherein the CE microparticles exhibit an oil absorption rate of at least 0.5 g/g according to ASTM D281 using olive oil instead of castor oil.

25. The method of claim 1, wherein the CE microparticles exhibit a monomodal particle size distribution with a span in the range of 1.0 to 1.5.

26. The method of claim 1, wherein the CE comprises at least one of cellulose acetate, cellulose acetate butyrate, or cellulose acetate propionate, and
wherein the CE has an average degree of substitution for hydroxyl substituents (“DS_{OH}”) in the range of 0.6 to 2.8.

27. The method of claim 1, wherein the CE microparticles have a polydispersity index of less than 0.8 and a sphericity of less than 70 percent.

28. The method of claim 1, wherein the CE microparticles have an average BET surface area of at least 0.9 m²/g.

29. The method of claim 1, wherein the CE microparticles have bulk density in the range of 0.2 to 0.3 g/m³.

30. The method of claim 1, wherein the CE microparticles have an average smoothness of at least 10 percent.
